(19) United States
(12) Patent Application Publication
(10) Pub. No.: US 2026/0238864 A1
(43) Pub. Date: Aug. 13, 2026

(54) RADIATION IMAGING SYSTEM, RADIATION IMAGING APPARATUS, CONTROL APPARATUS, AND NON-TRANSITORY COMPUTER-READABLE STORAGE MEDIUM

(71) Applicant: CANON KABUSHIKI KAISHA, Tokyo (JP)

(72) Inventors: TOMOYASU MIZUNO, Tokyo (JP); TOMOYA SUZUKI, Tokyo (JP)

(21) Appl. No.: 19/465,906

(22) Filed: Jan. 30, 2026

(30) Foreign Application Priority Data
Feb. 10, 2025 (JP) 2025-020244

(51) Int. Cl. H04N 23/30; H04N 23/56; H04N 23/63; H04N 23/68; H04N 23/73

(52) U.S. Cl. CPC H04N 23/30; H04N 23/56; H04N 23/635; H04N 23/6812; H04N 23/73

(57) ABSTRACT

A radiation imaging system that captures a radiation image based on radiation, includes: a radiation imaging apparatus including a radiation detection device in which a plurality of regions are provided in an aligned manner, the plurality of regions including a plurality of imaging pixels for capturing the radiation image and a plurality of detection pixels for monitoring an irradiation dose of the radiation; a detection unit configured to obtain posture information that includes information of a rotation angle of the radiation imaging apparatus; and a control unit configured to specify a receptor field for use in monitoring of the irradiation dose, based on the posture information, the receptor field being formed by a combination of neighboring regions among the plurality of regions.

### BACKGROUND

### Field of the Technology

The present disclosure relates to a radiation imaging system, a radiation imaging apparatus, a control apparatus, and a non-transitory computer-readable storage medium.

### Description of the Related Art

Radiation imaging apparatuses provided with a radiation detection device (e.g., an X-ray flat panel detector or FPD) that performs automatic exposure control (hereinafter, AEC) have been put to practical use. Radiation imaging apparatuses of this type are used as medical diagnosis devices, nondestructive testing devices, and so forth. In general, a detection region targeted for AEC in a radiation detection device is set by a user in accordance with a region of interest (e.g., lung field) of a subject, which is a target site of radiation imaging. However, there are cases where the position of the set detection region shifts from the actual position of the region of interest of the subject due to a relative positional relationship between the subject and the radiation detection device. The occurrence of the positional shift between the detection region and the region of interest could possibly cause a decrease in the accuracy of AEC.

According to a radiography system described in Japanese Patent No. 5904681, a configuration is disclosed in which a displacement amount of a relative position between a radiation source and a radiation detection device is detected, a current relative position, which is a relative position at the current point in time, is specified based on the displacement amount, and a position of a detection region is decided in accordance with the specified current relative position.

However, according to the technique described in Japanese Patent No. 5904681, a case can arise where rotation of the radiation detection device cannot be detected, and the detection region to be used in AEC deviates from a region of interest. For example, in a mobile X-ray machine in which the radiation detection device is not fixed via a radiographic stand or the like, it can happen that the radiation detection device rotates while hidden by the subject. Although the central positions of the radiation source and the radiation detection device do not shift, the detection region that is used in AEC could possibly deviate from the region of interest due to the rotation of the radiation detection device.

### SUMMARY

The disclosure provides a technique that enables a detection region that is used in AEC to be specified efficiently without the detection region shifting from a region of interest, even in a state where a radiation imaging apparatus has rotated. That is to say, the disclosure provides a technique that accurately and efficiently specifies receptor fields, which are targeted for monitoring of an irradiation dose, in a region of interest of a subject.

According to one aspect of the present disclosure, there is provided a radiation imaging system that captures a radiation image based on radiation, comprising: a radiation imaging apparatus including a radiation detection device in which a plurality of regions are provided in an aligned manner, the plurality of regions including a plurality of imaging pixels for capturing the radiation image and a plurality of detection pixels for monitoring an irradiation dose of the radiation; a detection unit configured to obtain posture information that includes information of a rotation angle of the radiation imaging apparatus; and a control unit configured to specify a receptor field for use in monitoring of the irradiation dose, based on the posture information, the receptor field being formed by a combination of neighboring regions among the plurality of regions.

Features of the present disclosure will become apparent from the following description of embodiments with reference to the attached drawings. The following description of embodiments is described by way of example.

### BRIEF DESCRIPTION OF THE DRAWINGS

The accompanying drawings, which are incorporated in and constitute a part of the specification, illustrate embodiments of the present disclosure, and together with the description, serve to explain the principles of the embodiments.

FIG. 1 is a diagram showing an exemplary schematic configuration of a radiation imaging apparatus system according to an embodiment.

FIG. 2 is a diagram showing an exemplary configuration of a radiation imaging system of a mobile X-ray machine according to an embodiment.

FIG. 3 is a diagram showing an exemplary hardware configuration of a control apparatus according to an embodiment.

FIG. 4 is a diagram showing an exemplary configuration of a radiation imaging apparatus according to an embodiment.

FIG. 5 is a diagram for describing arrangements of receptor fields and detection pixels in the receptor fields according to an embodiment.

FIG. 6 is a diagram for describing directions of rotation angles and a criterion of a yaw angle according to an embodiment.

FIG. 7 is a diagram for describing rotation of receptor fields according to a first embodiment.

FIG. 8 is a diagram for describing arrangements of receptor fields according to the first embodiment, with rotation of the radiation imaging apparatus taken into consideration.

FIG. 9 is a diagram for describing coordinates in connection with rotation of the radiation imaging apparatus according to the first embodiment.

FIG. 10 is a diagram for describing coordinates in receptor fields according to the first embodiment, with rotation of the radiation imaging apparatus taken into consideration.

FIG. 11 is a flowchart showing imaging processing by the radiation imaging apparatus according to an embodiment.

FIG. 12 is a diagram for describing deletion of receptor fields according to an embodiment, with rotation of the radiation imaging apparatus taken into consideration.

FIG. 13 is a diagram for describing coordinates in connection with a movement and rotation of the radiation imaging apparatus according to a second embodiment.

FIG. 14 is a diagram for describing a change in the shape of receptor fields according to a fourth embodiment, with a movement of the radiation imaging apparatus taken into consideration.

### DESCRIPTION OF THE EMBODIMENTS

Hereinafter, embodiments will be described in detail with reference to the attached drawings. Note, the following embodiments are not intended to limit the scope of the claims. Multiple features are described in the embodiments, but it is not the case that all such features are required, and multiple such features may be combined as appropriate. Furthermore, in the attached drawings, the same reference numerals are given to the same or similar configurations, and redundant description thereof is omitted.

It is assumed that radiation according to a disclosed technique includes, for example, not only α rays, β rays, and γ rays that are beams generated by particles (including photons) discharged through radioactive decay, but also beams that have energy of the same level or more, such as X-rays, particle rays, cosmic rays, and so forth.

### First Embodiment

The following describes a radiation imaging apparatus of the present embodiment with reference to the drawings.

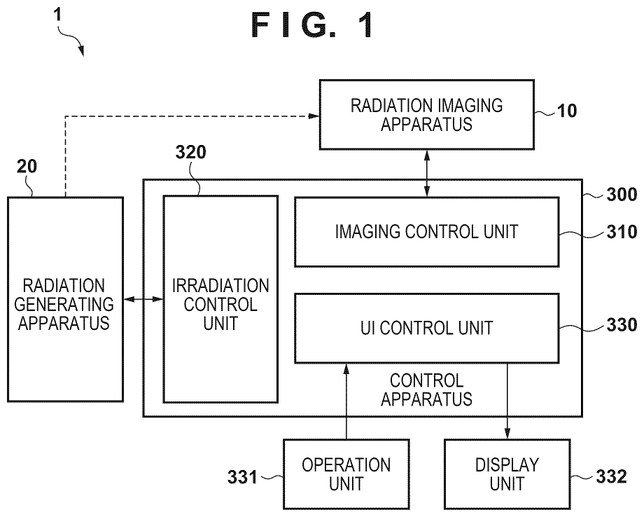

FIG. 1 is a functional block diagram showing an exemplary schematic configuration of a radiation imaging system 1 according to a first embodiment. The radiation imaging system 1 includes a radiation imaging apparatus 10, a radiation generating apparatus 20, and a control apparatus 300.

The radiation generating apparatus 20 includes a radiation tube that generates radiation, and irradiates a subject, such as a patient, with radiation. The radiation imaging apparatus 10 generates a radiation image based on radiation emitted by the radiation generating apparatus 20. The radiation imaging apparatus 10 includes, for example, a flat panel detector (FPD). Also, the radiation imaging apparatus 10 has a function of performing automatic exposure control (hereinafter, AEC). The details of the radiation imaging apparatus 10 will be described later. The radiation imaging system 1, which captures a radiation image based on radiation, includes: the radiation imaging apparatus 10 including a radiation detection device FPD that is provided with a plurality of imaging pixels for capturing a radiation image and a plurality of detection pixels for monitoring an irradiation dose of radiation; a detection unit that obtains posture information including information of a rotation angle of the radiation imaging apparatus 10; and the control apparatus 300 that specifies a receptor field, which is used in monitoring of an irradiation dose and includes one or more of the plurality of detection pixels, based on the posture information. The detection unit derives the posture information by obtaining an acceleration and an angular velocity from a sensor attached to the radiation imaging apparatus 10. The radiation imaging system 1 further includes a communication unit (communication I/F unit 305) that notifies the radiation imaging apparatus 10 of the receptor field specified by the control apparatus 30. Note that according to the configuration of the radiation imaging system 1 of FIG. 1, an exemplary configuration in which the detection unit and the control apparatus 300 are provided outside the radiation imaging apparatus 10 is shown; however, no limitation is intended by this example, and the detection unit and the control apparatus 300 may be provided inside the radiation imaging apparatus 10.

The control apparatus 300 is connectable to the radiation imaging apparatus 10 and the radiation generating apparatus 20. The control apparatus 300 includes an imaging control unit 310, an irradiation control unit 320, and a user interface (UI) control unit 330 as functional configurations. The imaging control unit 310, irradiation control unit 320, and user interface (UI) control unit 330 (hereinafter also referred to as UI control unit 330) in the control apparatus 300 may realize their respective functional configurations as a result of execution of a program by a central processing unit (CPU), or a dedicated or general-purpose processor, included in the control apparatus 300. Alternatively, they may be constituted by hardware, such as a field programmable gate array (FPGA) and an application specific integrated circuit (ASIC). The control apparatus 300 can also realize various types of functions by combining software processing by the processor and the program and hardware processing.

The imaging control unit 310 performs various types of control for radiation imaging by communicating with the radiation imaging apparatus 10. For example, the imaging control unit 310 transmits setting information of imaging conditions to the radiation imaging apparatus 10, and the radiation imaging apparatus 10 transmits image information and dose information to the imaging control unit 310. The imaging conditions include a stop determination threshold Dth and a set irradiation time period (hereinafter, backup time) Bt corresponding to a reaching target dose in AEC. Here, the dose information is an irradiation dose of radiation emitted by the radiation generating apparatus 20, and an incident dose denotes a dose that has reached the radiation imaging apparatus 10 out of the irradiation dose from the radiation generating apparatus 20. A dose obtained through processing for cumulating the incident doses (dose values) is a cumulative dose. The stop determination threshold Dth is threshold information that acts as a criterion in comparison between the reaching target dose and the cumulative dose. In automatic exposure control (AEC), irradiation is stopped when the cumulative dose has reached the reaching target dose. Also, the imaging conditions include receptor field information (ROI) that designates a receptor field used in AEC. The image information includes a radiation image obtained by the radiation imaging apparatus 10. The dose information includes, for example, a normal stop request that has been generated based on dose information obtained by the radiation imaging apparatus 10.

The irradiation control unit 320 sets irradiation conditions for radiation (a tube current (mA), a tube voltage (kV), backup time Bt, and so on) on the radiation generating apparatus 20 through communication with the radiation generating apparatus 20. Also, the irradiation control unit 320 transmits an irradiation control signal to the radiation generating apparatus 20 based on an exposure permission signal and the dose information (normal stop request) obtained from the radiation imaging apparatus 10. The irradiation control unit 320 causes the radiation generating apparatus 20 to start irradiation in response to the exposure permission signal, and causes the radiation generating apparatus 20 to stop irradiation in response to the normal stop request.

The UI control unit 330 controls inputting of information via an operation unit 331, and outputting of information via a display unit 332. For example, the imaging conditions and the irradiation conditions are input via the operation unit 331, and the result of radiation imaging (a radiation image) is output via the display unit 332. The operation unit 331 includes, for example, such input apparatuses as a keyboard, a pointing apparatus (e.g., a mouse or the like), and a touch panel. The display unit 332 includes, for example, a monitor, such as a liquid crystal display. The UI control unit 330 accepts the imaging conditions and the irradiation conditions that have been input by a user, such as a radiographer, with use of the operation unit 331. The accepted imaging conditions are transmitted to the radiation imaging apparatus 10 via the imaging control unit 310, and the accepted irradiation conditions are transmitted to the radiation generating apparatus 20 via the irradiation control unit 320.

The imaging control unit 310, irradiation control unit 320, and UI control unit 330 can work in coordination with one another through mutual communication. Note that although FIG. 1 illustrates the control apparatus 300 as one apparatus for simple explanation, the control apparatus 300 may be constituted by a plurality of apparatuses. For example, each of the imaging control unit 310, irradiation control unit 320, and UI control unit 330 may be an independent apparatus.

The control apparatus 300 controls operations of each of the radiation generating apparatus 20 and the radiation imaging apparatus 10 by connecting to the radiation generating apparatus 20 via wired communication, and connecting to the radiation imaging apparatus 10 via wired communication or wireless communication. As wired communication, for example, a local area network (LAN), such as Ethernet®, can be used, but the communication may be performed using other wired communication methods. Also, a configuration for wireless communication includes an antenna and a communication IC, for example. A circuit substrate that includes the communication IC executes communication processing of protocols that are based on a wireless LAN via the antenna. Note that a frequency band, a standard, and a method in the wireless communication are not limited in particular. For example, a near-field wireless method like near-field communication (NFC) or Bluetooth®, and a method such as ultra-wide band (UWB), can be used as the aforementioned wireless communication. Furthermore, the control apparatus 300 may be configured to be capable of using a plurality of wireless communication methods, and communication may be performed by selecting a method to be used in communication with the radiation imaging apparatus 10 as appropriate.

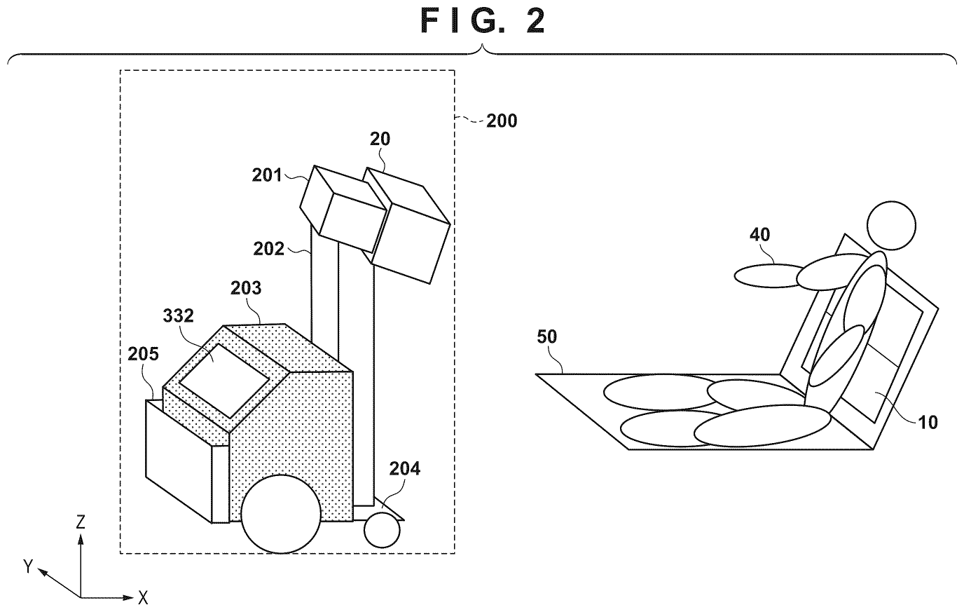

FIG. 2 is a conceptual diagram showing an exemplary configuration of a radiography system in a mobile X-ray machine of the present embodiment. A mobile X-ray machine 200 is a portable trolley provided with wheels and the like, and is equipped with the radiation generating apparatus 20. The mobile X-ray machine 200 is used, together with the radiation imaging apparatus 10, to perform radiography. To perform radiography, the radiation imaging apparatus 10 is situated at the back of a subject 40 who is on a bed 50. Based on radiation with which the radiation imaging apparatus 10 has been irradiated after the radiation has been irradiated by the radiation generating apparatus 20 and transmitted through the subject 40, the radiation imaging apparatus 10 generates a radiation-based image of the subject 40.

The mobile X-ray machine 200 is provided with a first arm 201 and a second arm 202 that support the radiation generating apparatus 20, in addition to the radiation generating apparatus 20. Also, the mobile X-ray machine 200 is provided with a housing 203, a base 204, and an FPD storage 205.

The mobile X-ray machine 200 includes a detection unit capable of obtaining a posture angle of the radiation generating apparatus 20. It is preferable for the detection unit to include components capable of obtaining a position and an angle, such as encoders, which are mounted inside the radiation generating apparatus 20, the first arm 201, and the second arm 202. Furthermore, it is preferable for the detection unit to be capable of detecting rotation of the mobile X-ray machine. Note that no limitation is intended by this; a posture angle and rotation may be detected using values that have been measured by attaching a hexaxial inertial measurement unit (IMU) including an acceleration sensor and a gyro sensor, or other known methods may be used. The posture angle may be represented by a Euler angle, or may be represented by a quaternion. However, as a quaternion is not a representation method that can be intuitively understood by a user, it is desirably converted into a Euler angle when displayed.

### <Regarding Configuration of Control Apparatus 300>

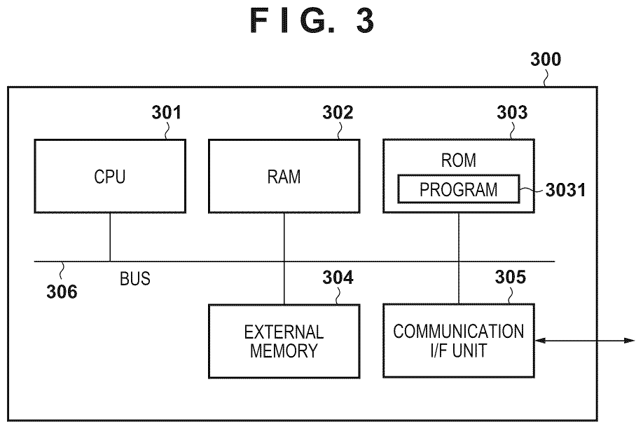

FIG. 3 is a diagram showing an exemplary configuration of the control apparatus 300. The control apparatus 300 includes a CPU 301 (central processing unit), a RAM (writable memory) 302, a read-only memory (ROM) 303, an external memory 304, a communication interface (I/F) unit 305, and a bus 306. The CPU 301, RAM 302, ROM 303, external memory 304, and communication I/F unit 305 are connected via the bus 306 in a mutually communicable manner.

The CPU 301 integrally controls operations of the control apparatus 300. The CPU 301 controls, for example, each configuration shown in FIG. 3 via the bus 306. The RAM 302 functions as a main memory, a working area, and the like for the CPU 301. When executing processing, the CPU 301 realizes various types of functional operations by loading a necessary computer program 3031, data, and the like from the ROM 303 to the RAM 302 and executing the computer program 3031 and the like. The ROM 303 stores the computer program 3031, data, and the like that are necessary for the CPU 301 to execute processing. Note that the computer program 3031, data, and the like may be stored in the external memory 304.

The external memory 304 is a large-capacity storage apparatus, and is realized by, for example, a hard disk apparatus, an IC memory, or the like. The external memory 304 stores, for example, various types of data, various types of information, and the like that are necessary when the CPU 301 carries out processing by executing the computer program 3031 and the like. Also, the external memory 304 stores, for example, various types of data, various types of information, and the like that have been obtained as a result of the CPU 301 carrying out processing by executing the computer program 3031 and the like. The communication I/F unit 305 takes charge of communication between the control apparatus 300 and the outside. The bus 306 connects the CPU 301, RAM 302, ROM 303, external memory 304, and communication I/F unit 305 in a mutually communicable manner.

Although the control apparatus 300 is provided as, for example, a dedicated built-in device, no limitation is intended by this, and it may be realized by a general-purpose information processing apparatus, such as a personal computer (PC) and a tablet terminal. Furthermore, as stated earlier, the control apparatus 300 may be constituted by a plurality of apparatuses; in this case, it is assumed that each of the plurality of apparatuses is configured in the above-described manner.

### <Regarding Configuration of Radiation Imaging Apparatus 10>

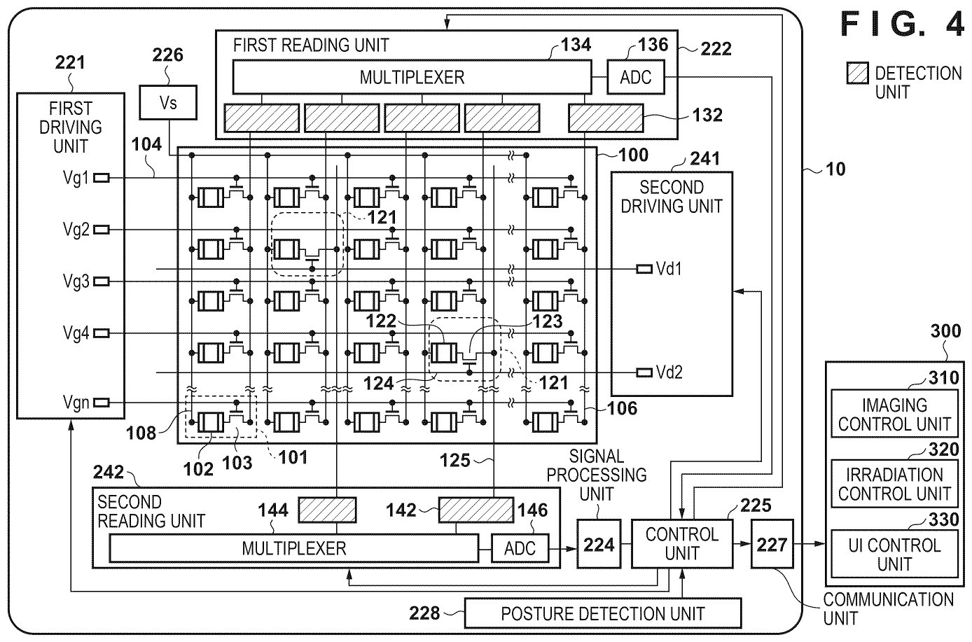

FIG. 4 is a diagram showing an exemplary configuration of the radiation imaging apparatus 10. The radiation imaging apparatus 10 includes a radiation detection device 100 (a sensor panel) in which a plurality of pixels are arrayed so as to compose a plurality of rows and a plurality of columns. The plurality of pixels arrayed in the radiation detection device 100 form an imaging region of the radiation detection device 100. The plurality of pixels include a plurality of imaging pixels 101 for obtaining a radiation image based on detected radiation, and a plurality of detection pixels 121 that function as detection pixels for dose detection, which are intended to monitor an irradiation amount of radiation. An imaging pixel 101 includes a conversion element 102 that converts radiation into an electrical signal, and a switch element 103 arranged between a column signal line 106 and the conversion element 102. A detection pixel 121 is configured similarly to an imaging pixel 101, and includes a conversion element 122 that converts radiation into an electrical signal, and a switch element 123 arranged between a detection signal line 125 and the conversion element 122.

The conversion elements 102 and 122 include a scintillator that converts radiation into light, and a photoelectric conversion element that converts light into an electrical signal. The scintillator is formed in a shape of a sheet so as to cover an imaging region formed by the plurality of imaging pixels 101, for example. Also, the conversion elements 102 and 122 may be replaced with conversion elements that convert radiation directly into electrical signals. The switch elements 103 and 123 are, for example, thin-film transistors (TFTs) with an active region that is constituted by a semiconductor like amorphous silicon or polycrystalline silicon. In the present embodiment, TFTs that use polycrystalline silicon are used as the switch elements 103 and 123.

The radiation imaging apparatus 10 includes a plurality of column signal lines 106, a plurality of detection signal lines 125, a plurality of driving lines 104, and a plurality of detection driving lines 124. Each of the plurality of column signal lines 106 and each of the plurality of detection signal lines 125 correspond to one column among the plurality of pixel columns in the imaging region of the radiation detection device 100. Each of the plurality of driving lines 104 and each of the plurality of detection driving lines 124 correspond to one line among the plurality of pixel lines in the imaging region of the radiation detection device 100. Here, a “column” matches a vertical direction in FIG. 4, and a “row” matches a horizontal direction in FIG. 4. A first driving unit 221 supplies driving signals to the driving lines 104, and a second driving unit 241 supplies driving signals to the detection driving lines 124.

The first driving unit 221 supplies driving signals to the imaging pixels 101 to be driven via the plurality of driving lines 104 in accordance with a control signal from a control unit 225. In the present embodiment, driving signals are signals for turning ON the switch elements 103 included in the imaging pixels 101 to be driven. The switch elements 103 are turned ON by high-level signals, and turned OFF by low-level signals. Therefore, such high-level signals are referred to as driving signals. As a result of supplying the driving signals to the imaging pixels 101, signals accumulated in the conversion elements 102 of these pixels are placed in a state where they can be read by a first reading unit 222. A first electrode of a conversion element 102 is connected to a first main electrode of a switch element 103, and a second electrode of the conversion element 102 is connected to a bias line 108. The bias line 108 extends in a column direction, and is connected commonly to the second electrodes of the plurality of conversion elements 102 arrayed in the column direction. A power source unit 226 supplies a bias voltage Vs to bias lines 108. In a plurality of imaging pixels 101 that compose one column, second main electrodes of the switch elements 103 are connected to one corresponding column signal line 106. In a plurality of imaging pixels 101 that compose one row, control electrodes of the switch elements 103 are connected to one corresponding driving line 104.

The plurality of column signal lines 106 are connected to the first reading unit 222. The first reading unit 222 includes detection units 132, a multiplexer 134, and an analog-digital (AD) converter 136. Each of the plurality of column signal lines 106 is connected to a corresponding detection unit 132 among the plurality of detection units 132 in the first reading unit 222. One column signal line 106 corresponds to one detection unit 132. A detection unit 132 includes, for example, a differential amplifier, and amplifies signals input from a column signal line 106. The multiplexer 134 selects the plurality of detection units 132 in a predetermined order, and supplies signals output from the selected detection unit 132 to the AD converter 136. The AD converter 136 converts the supplied analog signals into digital signals, and outputs the digital signals.

A detection pixel 121 is configured similarly to an imaging pixel 101. That is to say, a first electrode of a conversion element 122 is connected to a first main electrode of a switch element 123, and a second electrode of the conversion element 122 is connected to a bias line 108. A second main electrode of the switch element 123 is connected to a detection signal line 125. A control electrode of the switch element 123 is connected to a detection driving line 124. One or more detection pixels 121 (second main electrodes of the switch elements 123) aligned in the same column are connected to one detection signal line 125. The detection driving lines 124 are driven by the second driving unit 241. One or more detection pixels 121 (control electrodes of the switch elements 123) aligned in the same row are connected to one detection driving line 124.

The second driving unit 241 supplies driving signals to the detection pixels 121 to be driven via the plurality of detection driving lines 124 in accordance with a control signal from the control unit 225. As a result of supplying the driving signals to the detection pixels 121, signals accumulated in the conversion elements of these detection pixels 121 are placed in a state where they can be read by a second reading unit 242.

The plurality of detection signal lines 125 are connected to the second reading unit 242. The second reading unit 242 includes a plurality of detection units 142, a multiplexer 144, and an AD converter 146. Each of the plurality of detection signal lines 125 is connected to a corresponding detection unit 142 among the plurality of detection units 142 in the second reading unit 242. One detection signal line 125 corresponds to one detection unit 142. A detection unit 142 includes, for example, a differential amplifier, and amplifies signals input from a detection signal line 125. The multiplexer 144 selects the plurality of detection units 142 in a predetermined order, and supplies signals output from the selected detection unit 142 to the AD converter 146. The AD converter 146 converts the supplied signals into digital signals, and outputs the digital signals.

An output from the AD converter 146 in the second reading unit 242 is supplied to a signal processing unit 224 and processed by the signal processing unit 224. The signal processing unit 224 generates information related to radiation with which the radiation imaging apparatus 10 is irradiated based on the output from the AD converter 146 in the second reading unit 242, and outputs the information. The signal processing unit 224 obtains information of doses of radiation incident on the detection pixels 121 based on electrical signals that have been generated by the detection pixels 121 in accordance with irradiated radiation. The signal processing unit 224 may apply digital signal processing to signals obtained through digital conversion of signals from the detection pixels 121 (the output from the AD converter 146). The signal processing unit 224 detects a start of irradiation of the radiation imaging apparatus 10 with radiation based on the generated information. Alternatively, the signal processing unit 224 computes irradiation doses and a cumulative irradiation dose (reaching dose) of radiation based on the generated information.

The control unit 225 controls operations of each of the first driving unit 221, second driving unit 241, first reading unit 222, and second reading unit 242. The control unit 225 includes, for example, a CPU and a memory (a ROM or a RAM), and can execute a variety of types of processing by causing the CPU to execute programs stored in the memory.

The control unit 225 controls the first driving unit 221 and the first reading unit 222 based on information from the signal processing unit 224. Also, the control unit 225 controls a start and an end of exposure (accumulation of charges in the imaging pixels 101) based on information from the signal processing unit 224. Furthermore, the control unit 225 obtains, for example, dose information of radiation incident on the detection pixels 121 via the signal processing unit 224, and determines whether it is necessary to stop irradiation. In addition, the control unit 225 can control the second driving unit 241 independently from the first driving unit 221. In this way, the control unit 225 can obtain dose information from the outputs from the detection pixels 121 even during accumulation of charges in the imaging pixels 101.

A posture detection unit 228 derives posture information of the radiation imaging apparatus 10 by obtaining an acceleration and an angular velocity from a sensor attached to the radiation imaging apparatus 10. In the present embodiment, the posture information is a posture angle of the radiation imaging apparatus 10. A Euler angle may be output or a quaternion may be output as the posture angle. Furthermore, the sensor is constituted by a hexaxial inertial measurement unit (IMU) including an acceleration sensor and a gyro sensor. Note that the hexaxial IMU is an example; for example, a nine-axis IMU additionally including a geomagnetic sensor may be used, or one of the acceleration sensor, gyro sensor, and geomagnetic sensor may be used alone.

The radiation imaging apparatus 10 includes a communication unit 227 for carrying out communication with the control apparatus 300 (imaging control unit 310). The communication unit 227 includes one or both of a wired communication unit and a wireless communication unit. The communication unit 227 transmits information output from the control unit 225 to the control apparatus 300 with use of the wired communication unit or the wireless communication unit. For example, the communication unit 227 outputs information indicating whether it is necessary to stop irradiation, which has been decided by the control unit 225, to the control apparatus 300.

### <Description of Receptor Field>

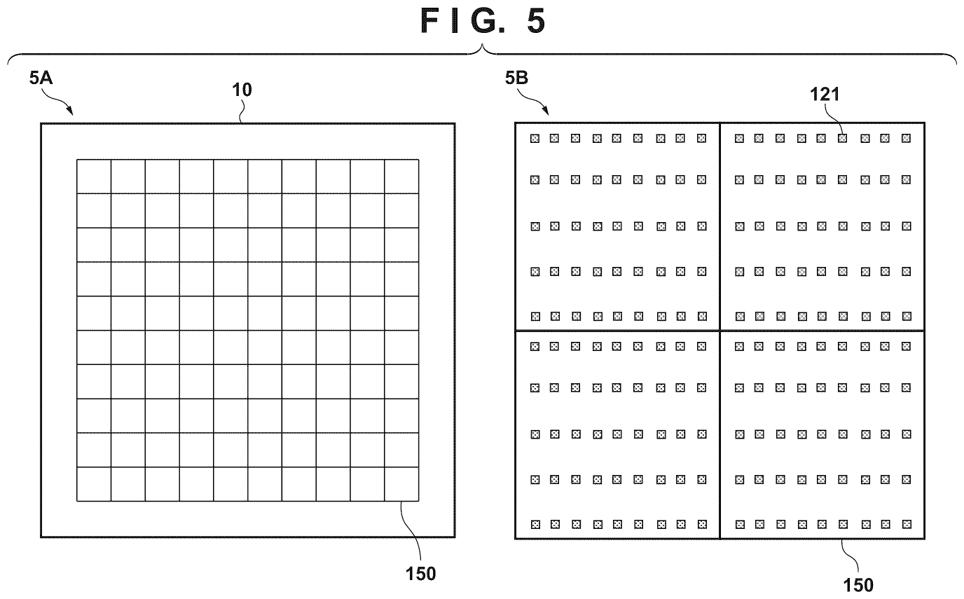

In an imaging region of the radiation detection device 100, one or more receptor fields that each include a plurality of detection pixels 121 are delimited. A receptor field is a region for detecting an irradiation dose of radiation during radiation imaging. An irradiation dose detected from a receptor field is decided based on irradiation doses detected by the detection pixels 121 included in this receptor field. FIG. 5 is a diagram for describing arrangements of receptor fields 150 and detection pixels 121 in the receptor fields 150. The receptor fields will be described using 5A of FIG. 5 as an example. As stated earlier, a receptor field 150 is a region for detecting an irradiation dose during imaging based on irradiation doses detected by the plurality of detection pixels 121 included in this receptor field. Although 5A of FIG. 5 shows an example in which 10× 10=100 receptor fields 150 are arranged in the imaging region of the radiation detection device 100, the plurality of receptor fields may be arranged in any manner. Note that when the receptor fields 150 are arranged symmetrically with respect to the center of the radiation imaging apparatus, AEC control that is not dependent on the direction of the radiation imaging apparatus 10 can be realized. Also, the receptor fields 150 are not limited to having a quadrilateral shape, such as a square shape and a rectangular shape, and may have any shape. For example, the shape of the receptor fields 150 may be a circular shape or an oval shape, or may be a shape that extends along the shape of the subject 40. In AEC, the generation of radiation by the radiation generating apparatus 20 is controlled based on irradiation doses obtained from the receptor fields 150 that have been selected in accordance with a radiographic site or the like. A user can freely select receptor fields to be used in dose detection in accordance with such conditions as the radiographic site. For example, the receptor fields to be used may be selected in accordance with the radiographic site, or the user may be able to select the receptor fields to be used at the time of radiation imaging. The receptor fields can be selected by the user with use of a GUI and the like, together with the control apparatus 300, operation unit 331, and display unit 332.

As shown in 5B of FIG. 5, in the present embodiment, a plurality of receptor fields are delimited in the radiation detection device 100 so that each receptor field includes one or more detection pixels 121. A receptor field 150 is constituted by detection pixels 121 that are arrayed one-dimensionally or two-dimensionally in the imaging region of the radiation detection device 100. An irradiation dose is detected in each receptor field 150. In the present embodiment, each of the plurality of receptor fields 150 includes the same number of detection pixels 121, and a total value of irradiation doses obtained from the detection pixels 121 inside a receptor field 150 can be used as an irradiation dose of the receptor field 150. In a case where the number of included detection pixels 121 varies among the plurality of receptor fields 150, it is necessary to use an average value obtained by dividing a total value of irradiation doses obtained from the respective detection pixels 121 inside a receptor field 150 by the number of the detection pixels 121. The plurality of receptor fields 150 are arranged one-dimensionally or two-dimensionally in the radiation imaging apparatus 10. As has been described using 5A of FIG. 5, receptor fields to be used in dose detection among the plurality of receptor fields 150 can be arbitrarily selected in accordance with such conditions as a radiographic site.

### <Relative Angle Between Radiation Generating Apparatus and Radiation Imaging Apparatus>

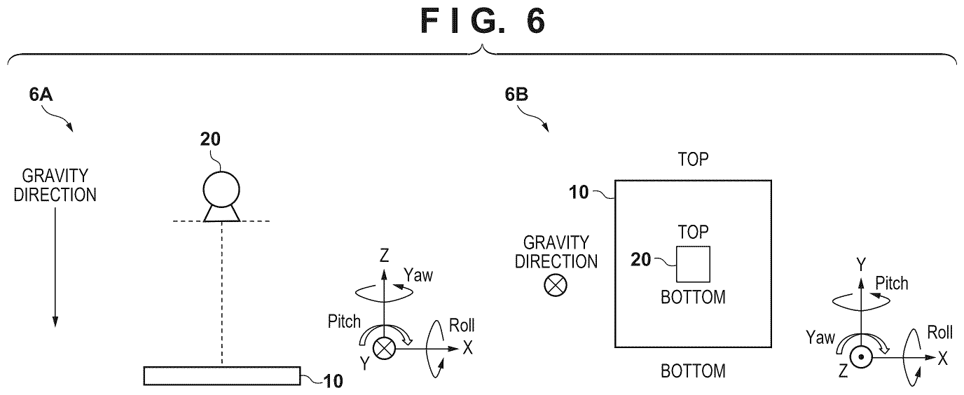

In the present embodiment, each of the radiation generating apparatus 20 and the radiation imaging apparatus 10 can detect a posture angle. FIG. 6 is a diagram for describing directions of rotation angles and a criterion of a yaw angle. A posture angle is indicated using triaxial angles, while considering rotation angles around the respective axes as a roll angle, a pitch angle, and a yaw angle around the X axis, Y axis, and Z axis, respectively, as shown in 6A of FIG. 6. Each of the X, Y, and Z axes is an output axis of the acceleration sensor. In this case, regarding the roll angle and the pitch angle, the rotation angles can be calculated using gravity as a criterion; however, regarding the yaw angle, the rotation angle cannot be calculated using gravity as a criterion. Therefore, it is necessary to calculate the yaw angle from an integrated value of angular velocities obtained from the gyro sensor. To meet a criterion of initial positioning of the yaw angle, it is necessary to bring the upper sides and the lower sides of the radiation imaging apparatus 10 and the radiation generating apparatus 20 in agreement, and prepare a criterion where the yaw angle is 0 degrees in a state where the upper sides and the lower sides are in agreement. In the radiography system, criteria of top and bottom are set for each of the radiation generating apparatus 20 and the radiation imaging apparatus 10, and the yaw angle is set in such a manner that it is 0 degrees in a state where the top and the bottom of both of the radiation imaging apparatus 10 and the radiation generating apparatus 20 are in agreement as shown in 6B of FIG. 6.

A relative angle between the radiation generating apparatus 20 and the radiation imaging apparatus 10 can be calculated from posture angles of the respective apparatuses. To derive the relative angle, calculation may be performed by obtaining Euler angle information from each apparatus, or an Euler angle may be output as the relative angle after obtaining quaternions and performing calculation. The posture detection unit 228 obtains posture information including information of a relative angle between the radiation generating apparatus 20, which performs irradiation, and the radiation imaging apparatus 10. The control unit 225 specifies (selects) receptor fields which are to be used in monitoring of an irradiation dose in automatic exposure control (AEC), and which include one or more of a plurality of detection pixels, based on the posture information. The control unit 225 specifies (selects) receptor fields in regions inside a region of interest of a subject with respect to which a radiation image is to be captured.

### <Selection of Receptor Fields>

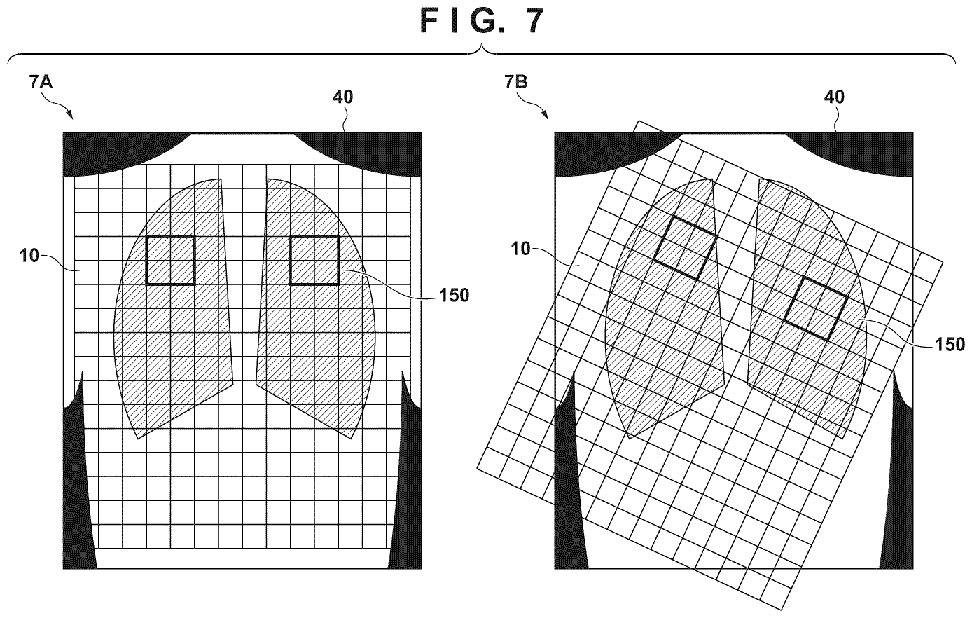

As stated earlier, a user can arbitrarily select receptor fields to be used in dose detection in accordance with such conditions as a radiographic site. For example, the receptor fields to be used may be selected in accordance with the radiographic site, or the user can select the receptor fields to be used at the time of radiation imaging. FIG. 7 is a diagram for describing rotation of receptor fields, and 7A of FIG. 7 shows an example in which a receptor field group is formed by selecting a plurality of receptor fields 150 while considering lung fields as target sites. When a user makes a selection, the selection is made within an ideal shooting range relative to the target sites while the radiation generating apparatus 20 and the radiation imaging apparatus 10 are an ideal position where they are facing each other (the centers thereof have no relative positional shift in the X and Y directions, and the relative angle is 0). However, in reality, a case can arise where they are not situated at the ideal position and the radiation imaging apparatus 10 is shifted. 7B of FIG. 7 is a diagram showing an example in which the radiation imaging apparatus 10 has rotated in the yaw angle direction. Although the radiation generating apparatus 20 can bring its direction in agreement with the subject 40 with use of, for example, an attached light guide, the radiation imaging apparatus 10 may rotate in a state where it is hidden behind the subject 40 and may be shifted from the ideal position, as in a state shown in 7B of FIG. 7. In this case, target sites of the lung fields, which are a region of interest, deviate from the selected receptor fields.

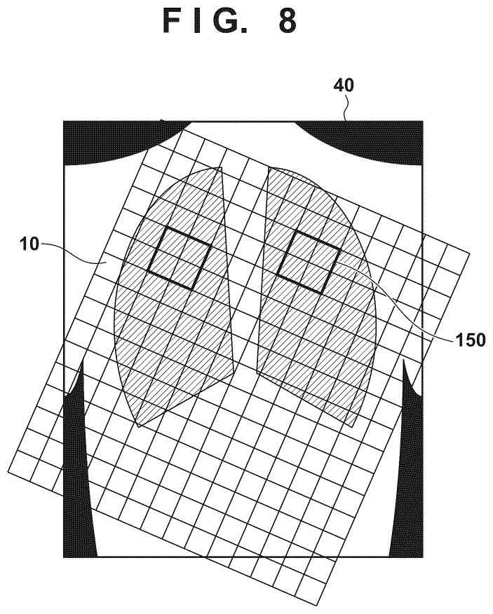

FIG. 8 is a diagram for describing arrangements of receptor fields with rotation of the radiation imaging apparatus 10 taken into consideration, and an example shown in FIG. 8 represents the receptor fields 150 that should be selected in a case where rotation of the radiation imaging apparatus 10 is taken into consideration. In order to carry out AEC with high accuracy, the receptor fields 150 should be selected so that the rotated receptor fields 150 are in a state where they are selected inside regions of the target sites of the lung fields, which are the region of interest, as shown in FIG. 8. In FIG. 8, the selected receptor fields 150 have not deviated from the target sites of the lung fields (portions with hatching), which are the region of interest, and are located inside the regions of the target sites, which are the region of interest.

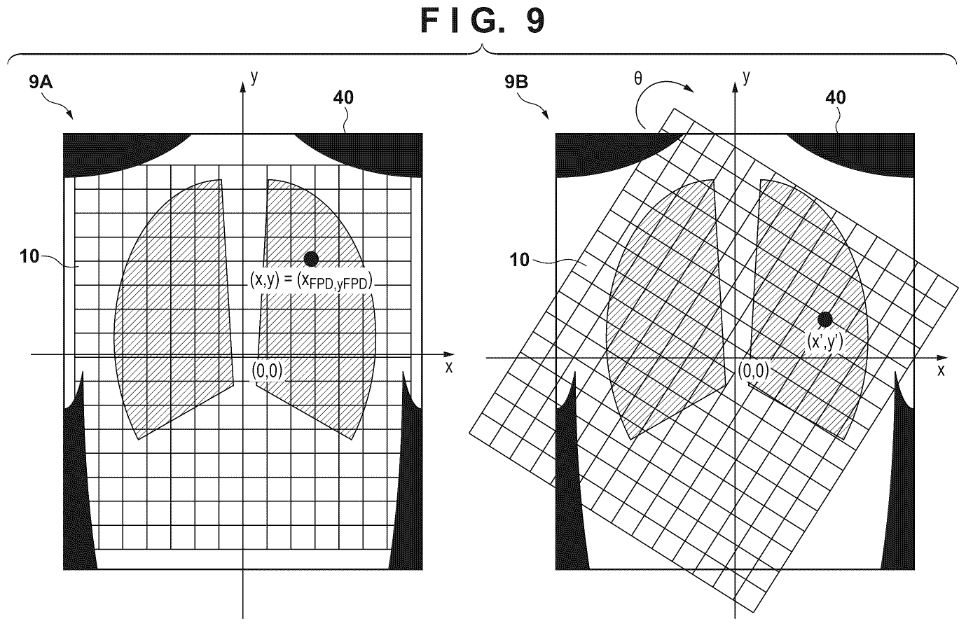

A method of selecting receptor fields in consideration of rotation of the radiation imaging apparatus 10 will be described using FIG. 9 as an example. FIG. 9 is a diagram for describing coordinates in connection with rotation of the radiation imaging apparatus 10, and 9A of FIG. 9 is a diagram showing a state where the radiation generating apparatus 20 and the radiation imaging apparatus 10 are facing each other and the arrangement state is ideal also in terms of an imaging range. In the case of a coordinate system (x_{FPD}, y_{FPD}) in which the central coordinates of the shooting range are (0, 0), the coordinates in the selected receptor fields are (x, y), and the central coordinates of the radiation imaging apparatus 10 are (0, 0), (x, y) and (x_{FPD}, y_{FPD}) match in the ideal state where the apparatuses face each other. Next, provided that the coordinates in the receptor fields in a state where the radiation imaging apparatus 10 has been rotated by θ in the yaw angle direction are (x′, y′) as shown in 9B of FIG. 9, the coordinates (x′, y′) in the receptor fields are indicated by formula 1.

$$\begin{matrix}[ Math . 1 ] & \\ [ \begin{matrix}{x}^{′} \\ {y}^{′}\end{matrix} ] = [ \begin{matrix}cos θ & - sin θ \\ sin θ & cos θ\end{matrix} ] [ \begin{matrix}x \\ y\end{matrix} ] & ( formula 1 )\end{matrix}$$

Here, the pre-rotation coordinates (x, y) are indicated by formula 2, with use of the coordinates (x′, y′) in the receptor fields after rotation by θ and a rotation angle (θ).

$$\begin{matrix}[ Math . 2 ] & \\ [ \begin{matrix}x \\ y\end{matrix} ] = {[ \begin{matrix}cos θ & - sin θ \\ sin θ & cos θ\end{matrix} ]}^{- 1} [ \begin{matrix}{x}^{′} \\ {y}^{′}\end{matrix} ] & ( formula 2 )\end{matrix}$$

In order for the coordinates (x′, y′) in the receptor fields after rotation to be in the target sites of the lung fields, which are the region of interest, it is necessary to select receptor fields given by formula 3 for the coordinate system (x_{FPD}, y_{FPD}) of the radiation imaging apparatus 10.

$$\begin{matrix}[ Math . 3 ] & \\ [ \begin{matrix}{x}_{FPD} \\ {y}_{FPD}\end{matrix} ] = {[ \begin{matrix}cos θ & - sin θ \\ sin θ & cos θ\end{matrix} ]}^{- 1} [ \begin{matrix}{x}^{′} \\ {y}^{′}\end{matrix} ] & ( formula 3 )\end{matrix}$$

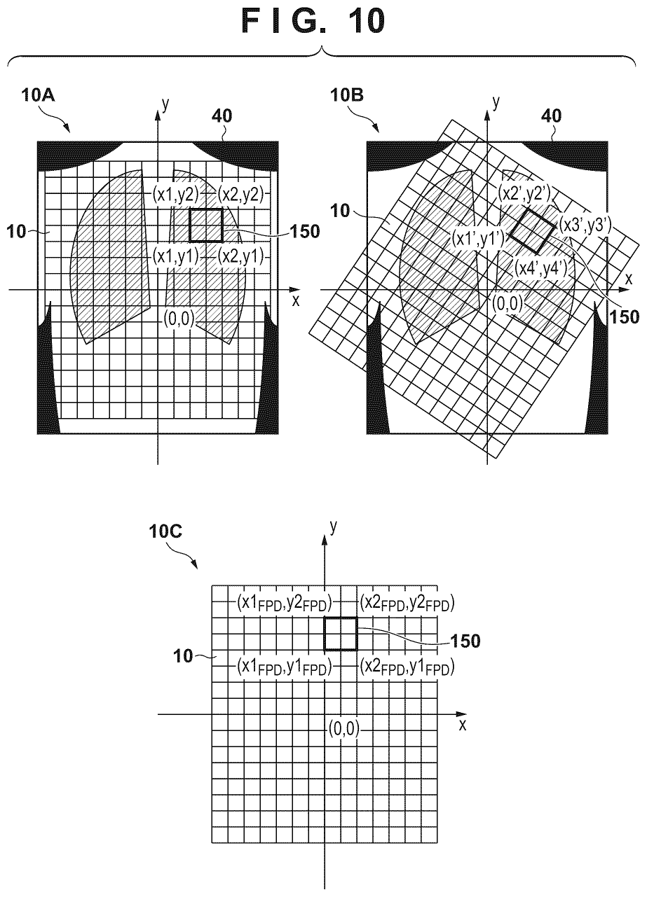

FIG. 10 is a diagram for describing coordinates in receptor fields with rotation of the radiation imaging apparatus taken into consideration, and is a diagram showing an example of a case where AEC is performed using a receptor field group after selecting a plurality of receptor fields. The coordinates of four corners of selected receptor fields are assumed to be (x1, y1), (x1, y2), (x2, y2), and (x2, y1) as shown in 10A of FIG. 10, and the coordinates of four corners after rotation are assumed to be (x1′, y1′), (x2′, y2′), (x3′, y3′), and (x4′, y4′) as shown in 10B of FIG. 10. Receptor fields (x1_{FPD}, y1_{FPD}), (x1_{FPD}, y2_{FPD}), (X2_{FPD}, y2_{FPD}), and (x2_{FPD}, y1_{FPD}) of the radiation imaging apparatus 10 are selected as shown in 10C of FIG. 10 by applying formula 3 so that all of the coordinates of the four corners after rotation are located inside the regions of the target sites of the lung fields, which are the region of interest. Note that in the present embodiment, a user can select the receptor fields shown in 10A of FIG. 10 via a GUI or the like before shooting. However, (x′, y′) after rotation may not completely match the coordinates selected by the user. In this case, (x′, y′) are selected so that their values are close to (x, y) selected by the user for the purpose of approaching the user's intention. That is to say, it is sufficient for the control unit 225 to preselect the receptor fields 150 of the region of interest based on the coordinate system ((x1_{FPD}, y1_{FPD}), (X1_{FPD}, y2_{FPD}), (x2_{FPD}, y2_{FPD}), and (x2_{FPD}, y1_{FPD})) in accordance with an angle included in the posture information, so that the coordinate information ((x1′, y1′), (x2′, y2′), (x3′, y3′), and (x4′, y4′)) of the receptor fields to which the coordinate conversion was applied based on the posture information is located inside a region of the region of interest.

The control unit 225 determines that the receptor fields have been specified in the region of interest in a case where the position information of the receptor fields after the coordinate conversion using the posture information is included in the region of interest, and determines that the receptor fields have been specified outside regions in the region of interest in a case where the position information of the receptor fields after the coordinate conversion using the posture information is not included in the region of interest.

### <Imaging Control>

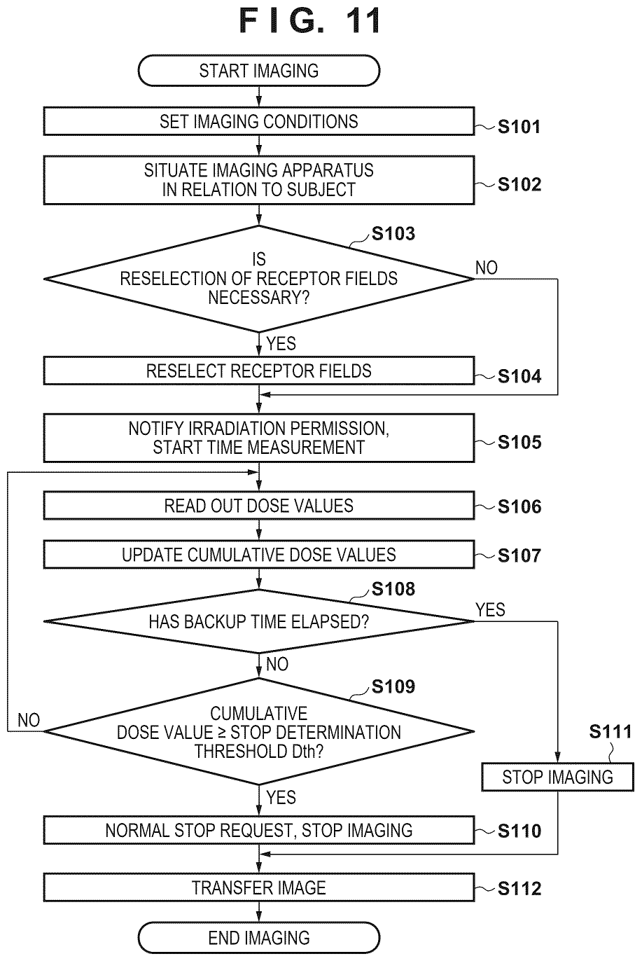

FIG. 11 is a flowchart showing imaging processing by the radiation imaging apparatus 10 according to an embodiment. In step S101, the control unit 225 of the radiation imaging apparatus 10 communicates with the control apparatus 300, and sets various types of information. Examples of the set information include a tube voltage and a tube current in a radiation tube, a stop determination threshold Dth, a set irradiation time period (backup time) Bt, and receptor field information (ROI information), as imaging condition information (irradiation condition information). Receptor fields to be used in AEC are selected based on the receptor field information. Note that the control unit 225 may receive a target dose Dref indicated by a dose index from the control apparatus 300, convert the target dose Dref into the stop determination threshold Dth (a signal value corresponding to the target dose), and set the stop determination threshold Dth. With this configuration, a user can designate a target dose with use of the dose index.

In step S102, the radiation imaging apparatus 10 is situated aligned with an imaging position of the subject. This is a process of situating the radiation imaging apparatus 10 between the subject 40 and the bed 50 in the example of FIG. 2.

In step S103, the control unit 225 determines whether it is necessary to reselect receptor fields. In the present embodiment, the control unit 225 checks whether the radiation generating apparatus 20 and the radiation imaging apparatus 10 are opposing each other in a state where the relative angle is 0 degrees. In a case where the relative angle is not zero degrees (0 degrees) (step S103—YES), processing proceeds to step S104; in a case where the relative angle is zero degrees (0 degrees) (step S103—NO), processing proceeds to step S105.

In step S104, the control unit 225 reselects receptor fields. In the present embodiment, receptor fields are reselected based on the relative angle. While taking rotation of the radiation imaging apparatus 10 and the like into consideration, the control unit 225 reselects receptor fields at positions that have been specified to approach the positions of the receptor fields in the region of interest selected in step S101, with use of the aforementioned formula 3. After the specified receptor fields have been reselected, processing proceeds to step S105. In a case where the angle (relative angle) included in posture information is not zero degrees, the control unit 225 reselects receptor fields so as to approach the positions of preselected receptor fields in the region of interest based on the receptor field information.

In step S105, the control unit 225 starts a preparation for receiving irradiation in response to a start request signal received from the control apparatus 300. Then, once the preparation for receiving irradiation has been completed, an exposure permission signal is switched from a Lo level to a Hi level. This exposure permission signal is transmitted to the radiation generating apparatus 20 via the irradiation control unit 320, and the radiation generating apparatus 20 starts to generate radiation. Also, at a timing when the exposure permission signal is switched to the Hi level, the control unit 225 starts to accumulate charges in the imaging pixels 101 by controlling the first driving unit 221, and starts time measurement with use of an internal timer. Consequently, radiation imaging is started. Hereinafter, processing after the start of radiation imaging will be described.

In step S106, the control unit 225 obtains dose values of one or more selected receptor fields 150 corresponding to the receptor field information by driving the second driving unit 241. As stated earlier, the dose values obtained from the receptor fields 150 are binned dose values of the plurality of detection pixels 121.

In step S107, under instruction from the control unit 225, the signal processing unit 224 executes binning processing and processing for cumulating dose values in the horizontal direction (row), and updates cumulative dose values.

In step S108, the control unit 225 determines whether the timing indicated by the internal timer has reached the backup time Bt, that is to say, whether the backup time Bt has elapsed. In a case where it has been determined that the timing indicated by the internal timer has reached the backup time Bt (step S108—YES), processing proceeds to step S111, and the control unit 225 stops imaging. At this time, the radiation generating apparatus 20 stops radiation in accordance with the backup time Bt included in the irradiation conditions. The control unit 225 reads out signals from the imaging pixels 101 by controlling the first driving unit 221. Then, in step S112, the control unit 225 transfers the signals that have been read out from the imaging pixels 101 as a radiation image to the control apparatus 300. Note that information indicating that radiation has been stopped in the set irradiation time period may be appended to this radiation image.

On the other hand, in a case where the timing indicated by the internal timer has not reached the backup time Bt (step S108—NO), the control unit 225 causes processing to proceed to step S109. In step S109, the control unit 225 compares the cumulative dose values from the receptor fields 150 with the stop determination threshold Dth. In a case where it has been determined that the cumulative dose values are not equal to or larger than the stop determination threshold Dth (step S109—NO), the control unit 225 causes processing to return to step S106. On the other hand, in a case where it has been determined that the cumulative dose values are equal to or larger than the stop determination threshold Dth (step S109—YES), the control unit 225 causes processing to proceed to step S110. For example, the determination in step S109 results in YES in a case where all of the cumulative dose values of the selected receptor fields 150 have exceeded the stop determination threshold Dth. Alternatively, the determination in step S109 may result in YES in a case where at least one of all of the cumulative dose values of the selected receptor fields 150 has exceeded the stop determination threshold Dth. In step S110, the control unit 225 transmits a normal stop request (AEC stop request) to the control apparatus 300. Then, the control unit 225 reads out signals from the imaging pixels 101 by controlling the first driving unit 221, and ends imaging. The signals that have been read out from the imaging pixels 101 are transferred to the control apparatus 300 as a radiation image in step S112. Note that information indicating that radiation has been stopped normally may be appended to this radiation image. The radiation image transferred to the control apparatus 300 is displayed on the display unit 332 for diagnosis, or is used in dose management.

As described above, according to the present embodiment, receptor fields are reselected so as to be located inside a region of imaging target sites (a region of interest) based on the relative angle of posture angle information of the radiation generating apparatus 20 and the radiation imaging apparatus 10. In this way, for example, in a case where receptor fields have deviated from the imaging target sites that were selected based on settings due to unintended rotation when situating the radiation imaging apparatus in radiation imaging, receptor fields can be reselected so as to be in the imaging target sites, and the accuracy of AEC is improved. Furthermore, it is possible to use a general method of selecting receptor fields (e.g., designation of receptor fields by a user, and selection of receptor fields in accordance with the imaging target sites), and appropriate receptor fields can be used in AEC without imposing a special burden on designation and selection of receptor fields.

Note that although the relative angle between the radiation generating apparatus 20 and the radiation imaging apparatus 10 is used in reselection of receptor fields in the above description, a posture angle of the radiation imaging apparatus 10 alone may be used. This case is based on the precondition that shooting is carried out when the radiation generating apparatus 20 is at an initial position relative to the subject 40 in a state where initial positioning of the radiation generating apparatus 20 and the radiation imaging apparatus 10 has been performed in advance. Alternatively, in a state where the posture angle of the radiation imaging apparatus 10 at an ideal shooting position has been decided, calculation is performed using an angle of shift from this posture angle.

Furthermore, although a yaw angle is used as an example of an angle used in reselection of receptor fields in the above description, the angle used in reselection of receptor fields is not limited to the yaw angle, and may be a posture angle related to a roll angle or a pitch angle. In this case, receptor fields can be reselected by calculating a shift from an ideal position that is based on a relative angle represented by the roll angle or the pitch angle. However, when there is a deviation from an ideal angle represented by the roll angle or the pitch angle, there is a possibility that a depth direction relative to an irradiation direction of radiation changes, thereby influencing the detection accuracy of sensors. In this case, it is sufficient to perform imaging at an angle close to the ideal angle by, for example, adjusting the angle of the bed 50 in advance.

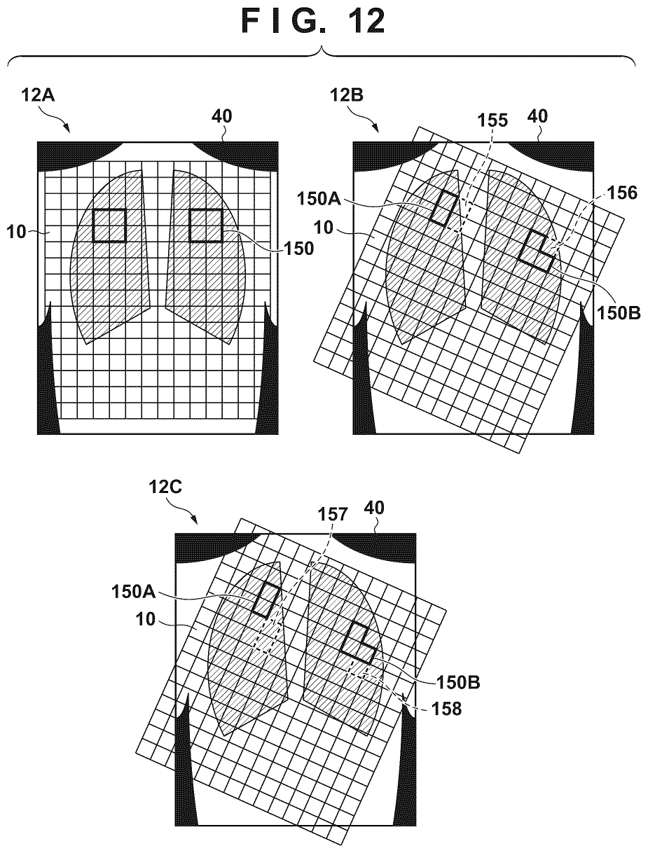

Furthermore, although the above description has been provided using an example in which receptor fields are reselected so that their values are close to (x, y) selected by a user via a GUI or the like, in a case where a receptor field group has been formed by selecting a plurality of receptor fields as shown in 12A of FIG. 12, it is permissible to, for example, change the shape of the receptor field group by deleting only receptor fields that have deviated from the target sites after rotation as shown in 12B of FIG. 12. In a case where the receptor fields (receptor field group) that were selected in a state where there is no shift in the relative angle have deviated from the region of interest due to coordinate conversion that is based on posture information, the control unit 225 changes the shape of the receptor fields (receptor field group) so that they are located inside a region of the region of interest. The control unit 225 deletes, from the receptor fields (receptor field group) 150 constituted by the plurality of detection pixels, a region that deviates from the region of interest (target sites), thereby changing the shape of these receptor fields (receptor field group) 150. For example, the shape of the receptor field group may be changed as indicated by 150A and 150B by deleting the receptor fields 155 and 156 that deviate from the target sites as shown in 12B of FIG. 12.

In the region of interest (inside the regions of the target sites), the control unit 225 may add regions (receptor fields 157 and 158) that have the same area as the deleted regions (receptor fields 155 and 156) to the receptor fields 150A and 150B that have been changed in shape, thereby changing the shape of these receptor fields (150A+157, 150B+158). It is sufficient for the control unit 225 to change the shape of the receptor field group so that the area of the receptor field group including the plurality of receptor fields before the shape change is the same as the area of the receptor field group including the plurality of receptor fields after the shape change. For example, as shown in 12C of FIG. 12, it is permissible to add the receptor fields 157 and 158 with areas equivalent to the deleted receptor fields 155 and 156 to the receptor fields 150A and 150B after the shape change in the region of interest (inside the regions of the target sites). In this case, the area of the receptor fields before deletion is equal to the area of the receptor fields after adding, to the receptor fields 150A and 150B that have been changed in shape, the receptor fields (receptor fields 157 and 158) with the same area as the deleted regions (receptor fields 155 and 156).

In a case where the area of the receptor fields after the shape change is smaller than the area of the receptor fields before the shape change, or in a case where the area of the receptor fields after the shape change is larger than the area of the receptor fields before the shape change, the control unit 225 may change the stop determination threshold Dth so that the correspondence relationship between the cumulative dose values detected in the receptor fields and the stop determination threshold Dth for stopping irradiation is the same (the timing to stop irradiation is the same) before and after the shape change.

For example, in a case where the area of the receptor fields after the shape change is smaller than the area of the receptor fields before the shape change, the control unit 225 may increase the stop determination threshold Dth so that the correspondence relationship between the cumulative dose values detected in the receptor fields and the stop determination threshold Dth is the same before and after the shape change.

Alternatively, in a case where the area of the receptor fields after the shape change is larger than the area of the receptor fields before the shape change, the control unit 225 may reduce the stop determination threshold Dth so that the correspondence relationship between the cumulative dose values detected in the receptor fields and the stop determination threshold Dth is the same before and after the shape change.

A radiation imaging system 1 that captures a radiation image based on radiation includes: a radiation imaging apparatus 10 including a radiation detection which includes an imaging region IR divided into a plurality of regions, and which includes a plurality of imaging pixels for capturing the radiation image and a plurality of detection pixels for monitoring an irradiation dose of the radiation in the imaging region IR; a detection unit that obtains posture information that includes information of a position of the radiation imaging apparatus; and a control apparatus 300 that specifies, as one receptor field group, a plurality of receptor fields for use in monitoring of the irradiation dose, based on the posture information, the plurality of receptor fields being formed by a combination of the plurality of regions.

The control apparatus 300 may change the position of one receptor field group based on the posture information, by changing the number of regions in the combination. Also, the control apparatus 300 may change the position of one receptor field group based on the posture information, without changing the number of regions in the combination. Furthermore, the control apparatus 300 may change the position of one receptor field group based on the posture information, without changing the number of regions in the combination, and without changing the shape of the one receptor field group. When changing the position of one receptor field group, the control apparatus 300 changes the position of the one receptor field group so that the one receptor field group is located inside a region of a region of interest of a subject with respect to which the radiation image is to be captured. The posture information obtained by the detection unit may include information of a relative angle between a radiation generating apparatus 20, which performs irradiation, and the radiation imaging apparatus 10.

### Second Embodiment

The present embodiment differs from the first embodiment in that a relative position of the radiation generating apparatus 20 and the radiation imaging apparatus 10 can be obtained as posture information. The following describes the present embodiment with a focus on differences from the first embodiment.

In the present embodiment, the radiation generating apparatus 20 of FIG. 2 includes a shooting apparatus (not shown) capable of obtaining an image (optical image) of the radiation imaging apparatus 10. The shooting apparatus can be constituted by, for example, a camera attached to the radiation generating apparatus 20. The shooting apparatus is attached to the radiation generating apparatus 20 so that the direction of shooting performed by the shooting apparatus matches the direction in which the radiation generating apparatus 20 performs irradiation at the time of imaging performed by the shooting apparatus. The control apparatus 300 can derive the relative position and the relative angle (rotation angle) of the radiation imaging apparatus 10 relative to the radiation generating apparatus 20 as posture information by obtaining a camera image (optical image) of the radiation imaging apparatus 10 from the shooting apparatus and analyzing the optical image.

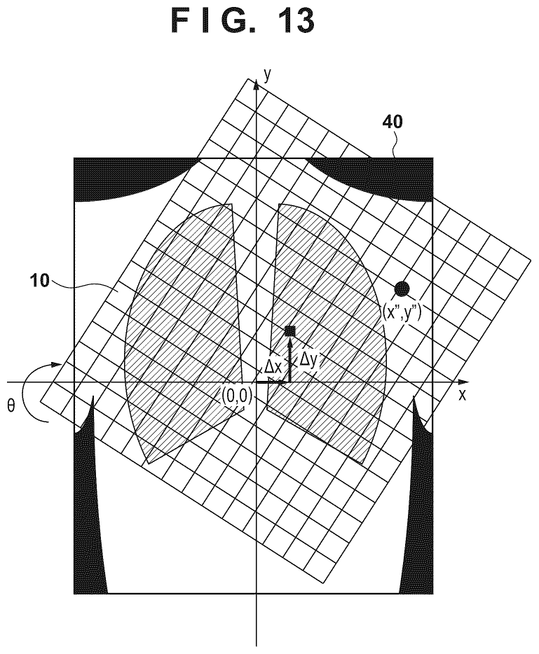

A method of selecting receptor fields in consideration of a movement and rotation of the radiation imaging apparatus 10 will be described using 9A of FIG. 9 and FIG. 13 as an examples. 9A of FIG. 9 is a diagram showing an ideal arrangement state as stated earlier. Also, FIG. 13 is a diagram for describing coordinates related to a movement and rotation of the radiation imaging apparatus 10 according to a second embodiment, and shows a state where the radiation imaging apparatus 10 has moved in the X direction and the Y direction by (Δx, Δy) and rotated by θ in the yaw angle direction. Provided that the coordinates in the receptor fields in the moved and rotated state are (x″, y″), the coordinates (x″, y″) in the receptor fields in the moved and rotated state are indicated by formula 4.

$$\begin{matrix}[ Math . 4 ] & \\ [ \begin{matrix}{x}^{″} \\ {y}^{″}\end{matrix} ] = [ \begin{matrix}cos θ & - sin θ \\ sin θ & cos θ\end{matrix} ] [ \begin{matrix}x + Δ x \\ y + Δ y\end{matrix} ] & ( formula 4 )\end{matrix}$$

Here, the coordinates (x, y) before the movement and rotation are indicated by formula 5, with use of the coordinates (x″, y″) in the receptor fields in the moved and rotated state and a rotation angle (θ).

$$\begin{matrix}[ Math . 5 ] & \\ [ \begin{matrix}x \\ y\end{matrix} ] = {[ \begin{matrix}cos θ & - sin θ \\ sin θ & cos θ\end{matrix} ]}^{- 1} [ \begin{matrix}{x}^{″} \\ {y}^{″}\end{matrix} ] - [ \begin{matrix}Δ x \\ Δ y\end{matrix} ] & ( formula 5 )\end{matrix}$$

In order for the coordinates (x″, y″) in the receptor fields after the movement and rotation to be in the target sites of the lung fields, which are the region of interest, it is necessary to select receptor fields given by formula 6 for the coordinate system (x_{FPD}, y_{FPD}) of the radiation imaging apparatus 10.

$$\begin{matrix}[ Math . 6 ] & \\ [ \begin{matrix}{x}_{FPD} \\ {y}_{FPD}\end{matrix} ] = {[ \begin{matrix}cos θ & - sin θ \\ sin θ & cos θ\end{matrix} ]}^{- 1} [ \begin{matrix}{x}^{″} \\ {y}^{″}\end{matrix} ] - [ \begin{matrix}Δ x \\ Δ y\end{matrix} ] & ( formula 6 )\end{matrix}$$

Imaging control of the second embodiment differs from the first embodiment in steps S103 and S104 in the flowchart of FIG. 11.

In step S103, the control unit 225 checks whether the relative position and the relative angle are zero (0) to confirm the necessity of reselection of receptor fields. This state where the relative position and the relative angle are zero (0) pertains to the X and Y directions, and the Z direction is not relevant. In a case where the relative position and the relative angle related to the X and Y directions are not zero (0), the control unit 225 determines that reselection is necessary (step S103—YES), and processing proceeds to step S104. On the other hand, in a case where the relative position and the relative angle are zero (0) in determination processing of step S103, the control unit 225 determines that reselection is unnecessary (step S103—NO), and processing proceeds to step S105.

In step S104, the control unit 225 reselects receptor fields based on the relative position and the relative angle. While taking the movement and rotation of the radiation imaging apparatus 10 into consideration, the control unit 225 reselects receptor fields so as to approach the positions of the receptor fields in the region of interest that were selected in step S101, with use of the aforementioned formula 6. After the receptor fields have been reselected, processing proceeds to step S105.

As described above, according to the present embodiment, receptor fields are reselected in radiographic target sites based on the relative position of and the relative posture angle between the radiation generating apparatus and the radiation imaging apparatus. In this way, even in a case where receptor fields have deviated from the imaging target sites that were selected based on settings due to the occurrence of the unintended positional shift or rotation of the radiation imaging apparatus 10, receptor fields can be reselected so that they are located inside the regions of the imaging target sites, which are the region of interest. In this way, the accuracy of AEC can be improved. Furthermore, it is possible to use a general method of selecting receptor fields (e.g., designation of receptor fields by a user, and selection of receptor fields in accordance with the imaging target sites), and appropriate receptor fields can be used in AEC without imposing a special burden on an operator in designation and selection of receptor fields.

Note that although analysis on a camera image (optical image) is used in deriving a relative position in the above description, no limitation is intended by this, and position information may be obtained by attaching sensors to each of the radiation generating apparatus 20 and the radiation imaging apparatus 10. For example, there is a method in which the acceleration sensor calculates a moving distance by using integration of accelerations, among others. Furthermore, a known position detection technique, such as a sensor of an ultra-wideband (UWB) method and Bluetooth, may be used.

### Third Embodiment

The present embodiment differs from the second embodiment in that, instead of using a general method of selecting receptor fields (e.g., designation of receptor fields by a user, and selection of receptor fields in accordance with the imaging target sites), receptor fields are selected automatically from a camera image (optical image) of the radiation imaging apparatus 10. The following describes the present embodiment with a focus on differences from the second embodiment.

In the first embodiment and the second embodiment, receptor fields are reselected on the precondition that an imaging range is in an ideal state as well. In the present embodiment, receptor fields (x, y) that act as a region of interest can be set by analyzing an examination subject shown in a camera image (optical image) and automatically recognizing radiographic sites, even if the imaging range is not in the ideal state.

Imaging control is different from the second embodiment in step S101 in the flowchart of FIG. 11. In step S101, the control unit 225 communicates with the control apparatus 300, and sets various types of information. Examples of the set information include a tube voltage and a tube current in a radiation tube, a stop determination threshold Dth, a set irradiation time period (backup time) Bt, and receptor field information (ROI information), as imaging condition information (irradiation condition information). In processing of step S101 of the present embodiment, the control apparatus 300 analyzes a camera image, and the control unit 225 sets obtained receptor field information based on the analysis by the control apparatus 300.

Thereafter, the flow is similar to that of the first embodiment or the second embodiment, and receptor fields are reselected from posture information with use of formula 3 or formula 6.

As described above, according to the present embodiment, receptor fields in radiographic target sites are selected based on a relative position of and a relative posture angle between the radiation generating apparatus 20 and the radiation imaging apparatus 10 that have been obtained through analysis on a camera image. In this way, even in a case where a subject can move in addition to an unintended positional shift or rotation of the radiation imaging apparatus 10, receptor fields can be selected at positions inside the regions of the imaging target sites, and the accuracy of AEC can be improved. Furthermore, as the control apparatus automatically designates receptor fields, appropriate receptor fields can be used in AEC without imposing a special burden on an operator in selection of receptor fields.

### Fourth Embodiment

The present embodiment will be described in relation to a configuration that differs from the second embodiment in that receptor fields are reselected based on the relative position of the radiation generating apparatus 20 and the radiation imaging apparatus 10. The following describes the present embodiment with a focus on differences from the second embodiment.

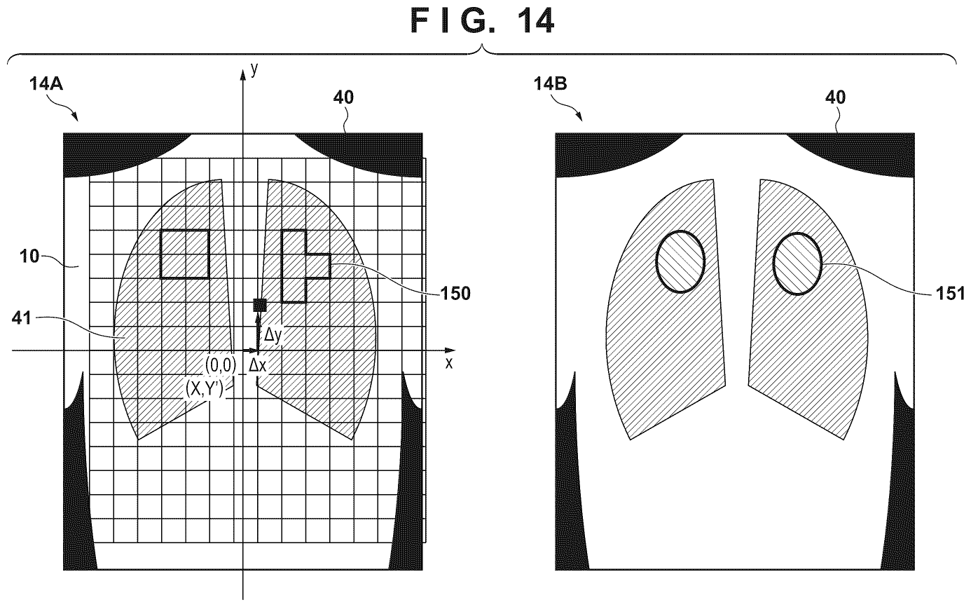

A method of selecting receptor fields in consideration of a movement of the radiation imaging apparatus 10 will be described using 10A of FIG. 10 and FIG. 14 as an example. As stated earlier, 10A of FIG. 10 is a diagram showing the coordinates (x1, y1), (x1, y2), (x2, y2), and (x2, y1) of four corners of selected receptor fields, and is a diagram showing that the radiation generating apparatus 20 and the radiation imaging apparatus 10 are facing each other and are in an ideal arrangement state in an imaging range as well. FIG. 14 is a diagram for describing a change in the shape of receptor fields that takes a movement of the radiation imaging apparatus 10 into consideration. Here, it is assumed that the coordinates of the radiation imaging apparatus 10 before the movement is (x, y), and a movement amount by which the radiation imaging apparatus 10 has moved in the X direction and the Y direction is (Δx, Δy). Also, provided that the post-movement coordinates of receptor fields in the moved state are (x′″, y′″), formula 7 holds.

$$\begin{matrix}[ Math . 7 ] & \\ [ \begin{matrix}{x}^{′′′} \\ {y}^{′′′}\end{matrix} ] = [ \begin{matrix}x + Δ x \\ y + Δ y\end{matrix} ] & ( formula 7 )\end{matrix}$$

Here, the pre-movement coordinates (x, y) are indicated by formula 8, with use of the post-movement coordinates (x′″, y′″) and the movement amount (Δx, Δy). The post-movement coordinates (x′″, y′″) are coordinates indicating a position that has deviated from a region of interest.

$$\begin{matrix}[ Math . 8 ] & \\ [ \begin{matrix}x \\ y\end{matrix} ] = [ \begin{matrix}{x}^{′′′} \\ {y}^{′′′}\end{matrix} ] - [ \begin{matrix}Δ x \\ Δ y\end{matrix} ] & ( formula 8 )\end{matrix}$$

In order for the post-movement coordinates (x′″, y′″) to be inside the regions of target sites of lung fields, which are the region of interest, it is necessary to select receptor fields given by formula 9 for the pre-movement coordinate system (x_{FPD}, y_{FPD}) of the radiation imaging apparatus 10.

$$\begin{matrix}[ Math . 9 ] & \\ [ \begin{matrix}{x}_{FPD} \\ {y}_{FPD}\end{matrix} ] = [ \begin{matrix}{x}^{′′′} \\ {y}^{′′′}\end{matrix} ] - [ \begin{matrix}Δ x \\ Δ y\end{matrix} ] & ( formula 9 )\end{matrix}$$

The present embodiment presents rearrangement of receptor fields that have deviated from the region of interest after the movement with use of formula 7.

In FIG. 14, a receptor field inside the region of interest that neighbors a receptor field that has deviated from the region of interest after the movement is reselected. In this case, although a receptor field group formed by selecting a plurality of receptor fields has a rectangular shape in 10A of FIG. 10, receptor fields that have been changed in shape may be arranged as shown in FIG. 14. In 14A of FIG. 14, if receptor fields move by a movement amount of (Δx, Δy), a case can arise where receptor fields 150 on the right side among receptor fields 150 on the left and right move to the outside of the region of interest (lung fields 41) and the receptor fields 150 do not fit in the region of interest. In this case, the shape of the receptor fields may be changed to, for example, the shape of the receptor fields 150 on the right side shown in 14A of FIG. 14 in consideration of the movement amount (Δx, Δy) in coordinate conversion. The example of the shape change of the receptor fields 150 is not limited to 14A of FIG. 14; for example, the shape may be changed to a circular shape or an oval shape as shown in 14B of FIG. 14. Note that among the receptor fields 150 on the left and right shown in 14A of FIG. 14, the receptor fields on the left side are located in the region of interest even if they have moved by (Δx, Δy); therefore they are not changed in shape, and the shape (rectangular shape) of the preselected receptor fields is maintained.

Although the lung fields 41 are the region of interest at the time of reselection in 14A of FIG. 14, priority levels (priority rankings) in rearrangement (reselection) may be set inside the region of interest. For example, the region of interest may be provided with regions for which a plurality of priority rankings are set in accordance with the site to be imaged and the direction of imaging, and the control unit 225 may reselect receptor fields based on the plurality of priority rankings. The control unit 225 may change the shape of receptor fields reselected based on the plurality of priority rankings so that the area of the receptor fields is larger than the area of the preselected receptor fields, and reselect the receptor fields that have been changed in shape.

For example, as shown in 14B of FIG. 14, the receptor fields 151 that have been changed in shape may be preferentially rearranged (reselected) near the upper lungs in the region of interest as regions with a high priority ranking. At this time, it is also possible to change the shape of receptor fields so that the area of the receptor fields 151 that have been preferentially rearranged (reselected) is larger than the area of the preselected receptor fields, and rearrange (reselect) the receptor fields that have been changed in shape. No limitation is intended by this example; when preferentially rearranging (reselecting) the receptor fields 151 that have been changed in shape near the upper lungs in the region of interest, it is also possible to change the shape of receptor fields so that the area of the receptor fields 151 that have been preferentially rearranged (reselected) is smaller than the area of the preselected receptor fields, and rearrange (reselect) the receptor fields that have been changed in shape.

Shooting control of the present embodiment differs from the second embodiment in processing of steps S103 and S104 in the flowchart of FIG. 11.

In step S103, in confirming the necessity of reselection of receptor fields, the control unit 225 checks whether the radiation generating apparatus 20 and the radiation imaging apparatus 10 are in a state where the relative position thereof is zero (0). In a case where the relative position is not zero (0) (step S103—YES), the control unit 225 determines that reselection is necessary, and processing proceeds to step S104; in a case where the relative position is zero (0) (step S103—NO), the control unit 225 determines that reselection is unnecessary, and processing proceeds to step S105.

In step S104, the control unit 225 reselects receptor fields based on the relative position. In a case where receptor fields have deviated from the region of interest, the control unit 225 rearranges (reselects) receptor fields in the above-described manner with use of the above-described formula 7.

As described above, according to the present embodiment, receptor fields are selected (reselected) in the radiographic target sites based on the relative position of the radiation generating apparatus 20 and the radiation imaging apparatus 10. Consequently, even on the occurrence of an unintended positional shift of the radiation imaging apparatus, receptor fields can be selected (reselected) in the imaging target sites, and the accuracy of AEC can be improved. Furthermore, it is possible to use a general method of selecting receptor fields (e.g., designation of receptor fields by a user, and selection of receptor fields in accordance with the imaging target sites), and appropriate receptor fields can be used in AEC.

As described above, according to a technique disclosed in each embodiment, a detection region used in AEC can be specified efficiently without the detection region shifting from a region of interest, even in a state where the radiation imaging apparatus has rotated. According to the technique disclosed in each embodiment, receptor fields targeted for monitoring of an irradiation dose can be specified accurately and efficiently in a region of interest of a subject.

According to the disclosed technique, receptor fields targeted for monitoring of an irradiation dose can be specified accurately and efficiently in a region of interest of a subject.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-020244, filed Feb. 10, 2025, which is hereby incorporated by reference herein in its entirety.

1-20. (canceled)

21. A radiation imaging system configured to perform automatic exposure control in which an irradiation dose is monitored and irradiation is stopped based on the irradiation dose, comprising:
a radiation imaging apparatus including a radiation detection device in which a plurality of receptor fields are provided;
a posture information acquisition unit configured to obtain posture information of the radiation imaging apparatus; and
a receptor-field specification unit configured to, based on the posture information, specify at least one of a position and a shape of a receptor field selected from among the plurality of receptor fields as a receptor field used for monitoring the irradiation dose in the automatic exposure control.

22. A radiation imaging apparatus configured to capture a radiation image based on radiation, comprising:
a radiation detection device in which a plurality of receptor fields are provided, a receptor field used for monitoring an irradiation dose in automatic exposure control being selectable from among the plurality of receptor fields;
a posture information acquisition unit configured to obtain posture information of the radiation imaging apparatus; and
a receptor-field specification unit configured to, based on the posture information, specify at least one of a position and a shape of the receptor field selected as the receptor field used for monitoring the irradiation dose in the automatic exposure control.

23. The radiation imaging apparatus according to claim 22, wherein the radiation detection device includes a plurality of imaging pixels for capturing the radiation image and a plurality of detection pixels for monitoring the irradiation dose, and each of the plurality of receptor fields includes one or more of the plurality of detection pixels.

24. The radiation imaging apparatus according to claim 22, wherein the receptor-field specification unit specifies at least one of the position and the shape of the receptor field within a region of interest of a subject.

25. The radiation imaging apparatus according to claim 24, wherein the receptor-field specification unit performs a coordinate transformation based on the posture information to specify at least one of the position and the shape of the receptor field such that the receptor field is located within the region of interest.

26. The radiation imaging apparatus according to claim 24, wherein, in a case where at least a part of the receptor field is outside the region of interest, the receptor-field specification unit changes the shape of the receptor field by performing at least one of:
deleting a portion of the receptor field;
adding a compensation portion; and
adjusting a stop determination threshold for stopping irradiation in accordance with a change in an area of the receptor field.

27. The radiation imaging apparatus according to claim 22, wherein the posture information acquisition unit derives the posture information using at least one of: an acceleration sensor, an angular velocity sensor, a geomagnetic sensor, and analysis of an optical image captured by an imaging device.

28. The radiation imaging apparatus according to claim 27, wherein the posture information includes a relative angle between a radiation generating apparatus and the radiation imaging apparatus.

29. The radiation imaging apparatus according to claim 22, wherein, in a case where a rotation angle included in the posture information is not zero degrees, the receptor-field specification unit specifies at least one of the position and the shape of the receptor field so as to approach a position of a preselected receptor field.

30. The radiation imaging apparatus according to claim 24, wherein the region of interest includes a plurality of subregions having respective priority rankings, and the receptor-field specification unit specifies at least one of the position and the shape of the receptor field based on the priority rankings.

31. The radiation imaging apparatus according to claim 22, further comprising a communication unit configured to notify an external apparatus of the receptor field specified by the receptor-field specification unit.

32. The radiation imaging apparatus according to claim 24, wherein the radiation detection device includes an imaging region divided into a plurality of subregions, and the receptor-field specification unit forms a receptor-field group by a combination of the plurality of subregions and specifies the receptor-field group such that the receptor-field group is located within the region of interest, by changing at least one of a number of subregions in the combination and a position of the receptor-field group based on the posture information.

33. A control apparatus configured to control a radiation imaging apparatus including a radiation detection device in which a plurality of receptor fields are provided, comprising:
a posture information acquisition unit configured to obtain posture information of the radiation imaging apparatus; and
a receptor-field specification unit configured to, based on the posture information, specify at least one of a position and a shape of a receptor field selected as a receptor field used for monitoring an irradiation dose in automatic exposure control.

34. The control apparatus according to claim 33, wherein the receptor-field specification unit specifies at least one of the position and the shape of the receptor field within a region of interest of a subject.

35. The control apparatus according to claim 34, wherein the receptor-field specification unit performs a coordinate transformation based on the posture information to specify at least one of the position and the shape of the receptor field such that the receptor field is located within the region of interest.

36. The control apparatus according to claim 34, wherein, in a case where at least a part of the receptor field is outside the region of interest, the receptor-field specification unit changes the shape of the receptor field by performing at least one of:
deleting a portion of the receptor field;
adding a compensation portion; and
adjusting a stop determination threshold for stopping irradiation in accordance with a change in an area of the receptor field.

37. The control apparatus according to claim 33, wherein the posture information acquisition unit derives the posture information using at least one of: an acceleration sensor, an angular velocity sensor, a geomagnetic sensor, and analysis of an optical image captured by an imaging device.

38. The control apparatus according to claim 37, wherein the posture information includes a relative angle between a radiation generating apparatus and the radiation imaging apparatus.

39. The control apparatus according to claim 33, wherein, in a case where a rotation angle included in the posture information is not zero degrees, the receptor-field specification unit specifies at least one of the position and the shape of the receptor field so as to approach a position of a preselected receptor field.

40. The control apparatus according to claim 34, wherein the radiation imaging apparatus includes an imaging region divided into a plurality of subregions, and the receptor-field specification unit forms a receptor-field group by a combination of the plurality of subregions and specifies the receptor-field group such that the receptor-field group is located within the region of interest, by changing at least one of a number of subregions in the combination and a position of the receptor-field group based on the posture information.

41. The control apparatus according to claim 33, further comprising a communication unit configured to notify the radiation imaging apparatus of the receptor field specified by the receptor-field specification unit.

42. The control apparatus according to claim 33, further comprising:
a display; and
a display control unit configured to cause the display to display receptor field information indicating at least one of the position and the shape of the receptor field specified by the receptor-field specification unit.

43. A control method for specifying a receptor field used for monitoring an irradiation dose in automatic exposure control in capturing a radiation image by a radiation imaging apparatus, the method comprising:
obtaining posture information of the radiation imaging apparatus; and
based on the posture information, specifying at least one of a position and a shape of a receptor field selected from among a plurality of receptor fields as the receptor field used for monitoring the irradiation dose in the automatic exposure control.

44. A non-transitory computer-readable storage medium storing instructions that, when executed by one or more processors, cause the one or more processors to perform a control method comprising:
obtaining posture information of a radiation imaging apparatus; and
based on the posture information, specifying at least one of a position and a shape of a receptor field selected from among a plurality of receptor fields as a receptor field used for monitoring an irradiation dose in automatic exposure control.
